(19) United States
(12) Patent Application Publication
(10) Pub. No.: US 2022/0249729 A1
(43) Pub. Date: Aug. 11, 2022

(54) LED LIGHT TROFFER WITH UV SANITIZED AIR RETURN

(71) Applicants: Timothy Hill, New York, NY (US); John Luk, Flushing, NY (US); Joel Gross, Brooklyn, NY (US); Ngoc-Thao Ly, Bronx, NY (US)

(72) Inventors: Timothy Hill, New York, NY (US); John Luk, Flushing, NY (US); Joel Gross, Brooklyn, NY (US); Ngoc-Thao Ly, Bronx, NY (US)

(73) Assignee: Lighting and Supplies, Inc., Brooklyn, NY (US)

(21) Appl. No.: 17/584,786

(22) Filed: Jan. 26, 2022

Related U.S. Application Data
(60) Provisional application No. 63/143,067, filed on Jan. 29, 2021.

(51) Int. Cl. A61L 9/20

(52) U.S. Cl. CPC A61L 9/20; A61L 2209/16; A61L 2209/12

(57) ABSTRACT

The present invention is directed to an improved combination LED light troffer and passive UVGI air chamber for providing both general lighting and disinfection of contaminated used air that is cleaned, treated, and purified before returning back to the HVAC system for recycling. The improvement including an internal and quiet UVGI air chamber that uses natural air convection from the HVAC system to take in air for sanitizing inside the UVGI air chamber for a brief amount of time, and then releasing it out to the air return of the HVAC system. The UVGI air chamber being operational only when the HVAC system is active and independent of the operation of the LED light troffer for general lighting. UVC light sources include germicidal fluorescent lamps, cold cathode lamps, excimer type lamps, and UVC type LED emitters in different UVC wavelengths.

### FIELD OF THE INVENTION

The present invention relates to an improved LED light troffer in combination with a constant and active UVGI air-sanitizer that automatically takes in air from a space up into the luminaire where the used air is cleaned by UVC light, and the purified air is returned into the HVAC system for eventual reuse in the conditioned space.

### BACKGROUND OF THE INVENTION

HVAC stands for Heating, Ventilation, and Air Conditioning. HVAC refers to the different systems used for moving air between indoor and outdoor areas, along with heating and cooling both residential and commercial buildings and the spaces found in these structures.

HVAC is an important part of residential structures such as single and multi-family homes, apartment buildings, hotels and senior living facilities, medium to large industrial and office buildings such as skyscrapers and hospitals, schools and colleges, dormitories, large meeting and concert halls, etc. where safe and healthy building environmental conditions are regulated with respect to temperature and humidity, using fresh air supplied by the outdoors.

Ventilating or Ventilation (the “V” in HVAC) is the process of exchanging or replacing outside air with inside air in any space to provide high indoor air quality which involves temperature control, oxygen replenishment, and removal of moisture, odors, smoke, heat, dust, harmful airborne bacteria and viruses, mold and spores, Volatile Organic Compounds or VOC's, carbon dioxide, and other gases. Ventilation can also remove any unpleasant smells and excessive moisture, introduces outside air, keeps interior building air circulating, and prevents stagnation of the interior air with fresh air supply.

With the SARS-CoV-2 coronavirus pandemic hitting the United States in early 2020, there became an immediate need and increased demand for Ultraviolet Germicidal Irradiation or UVGI products to help clean and sanitize the buildings, classrooms, and office spaces both safely and inconspicuously without interfering with our daily activities and allowing us all to perform our work and normal routines as before.

There is an UVGI return air grill vent system available in the market made by Halton and distributed by Nelbud Services Group that works with existing RTU (Roof Top Fixture) or MUA (Make Up Air) systems. It provides low pressure loss and promises not to impact RTU performance as it is designed to match airflow for the return air grill it replaces. The present invention is an improvement to this single UVGI return air grill product by including general use lighting from a combination troffer and UVGI return air cleaning fixture that can easily replace both standalone return air grills and standard LED lighting troffers offering the benefits of a multi-purpose and safe UVGI device.

Likewise, Halton makes a standalone UVGI Overhead Light fixture that is also distributed by Nelbud Services Group and is designed to fit into a standard 24″×48″ (2′×4′) ceiling grid. Multiple fixtures can be used throughout the space during unoccupied times to disinfect up to 99.99% of all exposed surfaces. Optional occupancy sensors and timers may be used with this dedicated UVGI overhead light troffer that is meant to be used along with conventional general use LED light troffers. The present invention is an improvement to this single UVGI Overhead Light by including a combination general use light troffer and UVGI sanitizing return air fixture that can easily replace both dedicated UVGI light fixtures and individual standard general use LED lighting troffers offering the benefits of a multi-purpose and safer improved combination of a general lighting troffer and UVGI air-sanitizing fixture in one.

There are also many air return troffers manufacturers out in the market now including the Airvola Recessed Air-Handling Troffer made by ILP, Inc.; Day-Brite/CFI FluxGrid LED recessed troffer by Signify; Cleanse Retrofit Troffer; Uventx UVC Air Disinfecting Panel, and many others. The Airvola and Day-Brite recessed troffers both have vent holes that allow stale air to enter up into the air return plenum space while the Cleanse Retrofit Troffer and Uventx UVC Air Disinfecting Panel both use fans to suck in used air that is trapped in replaceable HEPA and carbon activated filters that capture up to 99.97% of air particles, and then uses both UVA and UVC light or UVC respectively, to sanitize the filtered air to be recirculated back into the same space below with no provisions for adding in fresh air or heating and cooling the air.

Other UVGI systems use UVC light sources in the fresh air or return air sections of the HVAC system to destroy mold spores, bacteria, viruses and VOC's found on the air filters, cooling coils, and drain pans to prevent them from entering the inhabitable air spaces. But these systems are usually larger and more expensive to install and maintain versus the present invention of an improved UVGI air-sanitizing that is combined with a troffer light fixture. These systems could be used in conjunction with this invention to provide a multilayered approach to UV disinfection of building air.

Lastly, there are presently wall mounted upper room UVGI fixtures that are installed high up close to the ceiling on opposing walls that circulate the air within a room that continuously disinfect the air from bacteria and viruses that are carried into the UV field by convection currents and are deactivated or destroyed. This is achieved by the constant UVC rays being projected across the upper room air space. The UV light is typically baffled to prevent viewing from areas down below these upper room installed UVGI fixtures. However, these fixtures still present some safety concerns whenever someone may accidentally come into the UVC disinfection zone that is typically above 2.1-meters or about 7.0-feet from the ground.

There is presently no such product in the market that combines an improved LED light troffer and UVGI air-sanitizing return system in one fixture to safely and quietly clean and disinfect used contaminated air automatically, and then return the clean treated air back into the HVAC system like the present invention.

The main points of difference for the present invention versus what is presently out in the market at the time of this application include the simple deployment of the combination troffer and UV air return and disinfection luminaire by simply upgrading the lighting in the building and the HVAC system is upgraded now to have localized UV sanitation of return air.

Also, the UV sanitation is contained within the at least one UVGI air chamber without risk of exposure of the UV energy to occupants in the space below and above the ceiling.

Lastly, the building aesthetic is maintained without additional devices, grills, or grates in the user space that may disrupt the uniformity of the ceiling.

### SUMMARY OF THE INVENTION

The present invention relates to an improved combination LED light troffer and UVGI air-sanitizing fixture for use with new or existing HVAC systems. The UV light source can remain on at all times or can be manually turned-off, or may operate in synch when the HVAC system is active for energy saving purposes. The used air is treated, and then sent back to the return plenum and using ductwork, sends it back to the core HVAC system. This air is recycled as a fresh batch later by the HVAC system.

The subject invention works particularly with the air handling plenum space provided with the HVAC system. The present invention utilizes the return air flow from the conditioned space below to provide movement of air without the use of additional fans that can break down and make noise. The air handling plenum space helps to facilitate the air circulation for heating and air conditioning systems. The space between the structural ceiling and the dropped ceiling or under a raised floor is typically considered plenum versus a conventional large box connected to the HVAC ductwork, but this alternate space may also work to provide pathways for either the heated or air-conditioned return airflows from below the ceiling space.

The subject invention represents an improved LED troffer and UVGI air-sanitizing fixture that sucks up the used air from the rooms and spaces down below the ceiling back up into the return ducts. The air from the conditioned space collects in the return plenum and is drawn back into the main HVAC equipment through the return inlet often utilizing larger entire building air filters in standard ductwork for another round of heating or cooling where the air is recycled and treated continuously as long as the HVAC system is in operation.

There are at least the following unique aspects of the present invention. The present invention is essentially safe to use and operate, because the UVC germicidal light sources are self-contained inside at least one UV air chamber juxtaposed the recessed LED light troffer, and mounted up in the top ceiling area where the UVC light is not visible to anyone near the fixture. The used air is sucked into a return air chamber by natural convection created by the HVAC system and is slowed down to be cleaned by an adequate dose of UVC light, and then the disinfected and treated air is expelled out into the air handling plenum and returned back into the HVAC system. The choice and number of UVC LED light sources are chosen to emit enough UVC light to inactivate greater than 90% of airborne viruses and bacteria particles in the shortest amount of dosing time. This disinfection process is continuous as long as there is airflow created by an active HVAC system in operation with a visual inspection window that can be easily seen from below the fixture showing proof of activity and effectiveness that the UVC is in operation when the HVAC system is on.

The UVC light source(s) used in the combined air-sanitizing LED light troffer of the present design include UVC germicidal fluorescent tube lamps, or cold cathode UVC tubes, UVC excimer light sources in addition to UVC LEDs mentioned before. The wavelength of the UVC light may be 222 nm far UVC, 254 nm for germicidal fluorescent and cold cathode UVC lamps, and 250-285 nm for UVC LEDs. An example of a UVC LED linear light source that can be used in the present invention among other suppliers include the 24 LED DC module board Model Number XMD-FBx-LLxA that uses Seoul Viosys UVC LEDs and supplied by SETi.

The main object of the present invention is to provide an improved combination air-sanitizing LED light troffer upgrade to an HVAC system now with local UV disinfection of the returned air by using a target UV dose of 1,500 uW/cm² to all air passing through to the air return in an installed space from at least one on-board air chamber.

Another object of the present invention is to provide an improved combination air-sanitizing LED light troffer that includes quieter operation. The present invention is quiet with no active fans onboard, and instead relies on the natural convection airflow created by the HVAC system using the area above the finished ceiling as a ductless plenum air return circulation space. The present invention also offers increased energy savings, because it may only operate while the HVAC system is on and does not include the additional costs for noisy and short operating life electrical fans to be used.

Also, another object of the present invention is to provide an improved combination air-sanitizing LED light troffer without the regular need to replace dirt and dust collecting filters as compared to other units UVC sanitizing light fixtures. Unlike other standalone sanitizing air cleaners that use HEPA and other filters with UVC disinfecting light, the present invention is basically maintenance free without having to change out the filters at regularly scheduled intervals and at an additional post purchase cost.

It is yet another object of the present invention of an improved combination UVC air cleaning chamber and LED troffer light that will provide a small visible lens for each UVC air cleaning chamber for the visual inspection of the UV lamp operation from beneath the ceiling. In the event the UV lamps need to be replaced, the expired UV lamps can be easily replaced and maintained from below the ceiling. There is provided a power interlock switch for each UVC air cleaning chamber that will de-energize power to the UV lamps if any one of the two lamp change doors are opened for both safety from UV exposure and possible electrical shock.

Lastly, the UVGI device used in the present invention consisting of an improved combination UVC air cleaning chamber and LED troffer light can both work with or operate independent of each other. The air return capability of the present invention reduces the number of unsightly ceiling vents for a cleaner appearance. There are decorative air intake grills that provide aesthetic setting to any space motif. In addition, the UVC air cleaning chamber can operate continuously whenever the HVAC system is active, while the LED troffer light can operate on its own schedule offering the use of many troffer benefits and optional non-essential accessories such as integrated sensors for standalone occupancy, daylight dimming, emergency backup lighting, CCT tunability including dynamic tunable white and color control, power wattage adjustability, thermal foldback, lumen maintenance compensation, etc.

These and other aspects, features, and advantages of the present invention will become more readily apparent from the following attached drawings and the detailed description of the preferred embodiments.

### BRIEF DESCRIPTION OF THE DRAWINGS

The preferred embodiments of the invention will be described in conjunction with the appended drawings provided to illustrate and not to the limit the invention, where like designations denote like elements, and in which:

FIG. 1 shows a drawing of an existing UVGI air vent grill fixture.

FIG. 2 shows a drawing of an existing air-handling troffer.

FIG. 3 shows a drawing of a typical exploded view of the inside portion of HVAC air-duct showing a UVGI system used to clean the air to a building.

FIG. 4A shows a drawing of an existing high wall mounted upper air UVGI fixture.

FIG. 4B shows a picture of the air flow from two high wall mounted upper air UVGI fixtures shown in FIG. 4A used in a typical hospital room.

FIG. 4C is an alternate picture showing the air flow from a high wall mounted upper air UVGI fixture described in FIG. 4A, but now in a meeting room filled with occupants.

FIG. 4D is a picture of a space with one high wall mounted upper air UVGI fixture showing the minimum safe installation height of the upper air UVGI fixture and the maximum effective sterilization distance from the upper air UVGI fixture germicidal light source.

FIG. 5 shows how a room can be cleaned using only wall mounted upper air UVGI fixtures for air disinfection, and also shows how the same room can be cleaned using a combination of wall mounted upper air UVGI fixtures and ceiling mounted UVGI fixtures for both air and surface disinfection.

FIG. 6A shows both a front and isometric view of the present invention of an improved LED light troffer combined with a UVGI air-sanitizing from the ground showing typical air intake slots or vents, etc.

FIG. 6B shows both a rear and isometric view of the present invention of an improved LED light troffer combined with a UVGI air-sanitizing from the ground showing typical air intake slots or vents, etc.

FIG. 6C show side views of the present invention of an improved LED light troffer combined with a UVGI air-sanitizer with the end plate present and also removed showing typical air intake slots or vents and internal parts.

FIG. 7A shows an inside view of an exemplary embodiment of a UVGI air chamber of the present invention showing air entering from the intake slots or vents from FIG. 6A.

FIG. 7B shows a cross-sectional transparent end view of a typical embodiment of a UVGI air chamber of the present invention showing air passing through the slots or vents from FIGS. 6A-6C.

FIG. 8 shows a picture of the disinfection and purification process taking place within the UVGI air chamber of FIG. 7A where the used contaminated air enters and the clean treated air exits the LED light troffer and UVGI air-sanitizing fixture.

FIG. 9A shows a top view above the ceiling of the now clean and treated return air exiting the UVGI air chamber as described in FIG. 8 of the present invention next entering the return air circulation and plenum space, and then back to the main HVAC system for recycling using a standard ductwork and air grill.

FIG. 9B shows a bottom view below the ceiling of the now clean and treated return air exiting the UVGI air chamber as described in FIG. 8 of the present invention next entering the return air circulation and plenum space, and then back to the main HVAC system for recycling using a standard ductwork and air grill.

### LIST OF NUMERALS

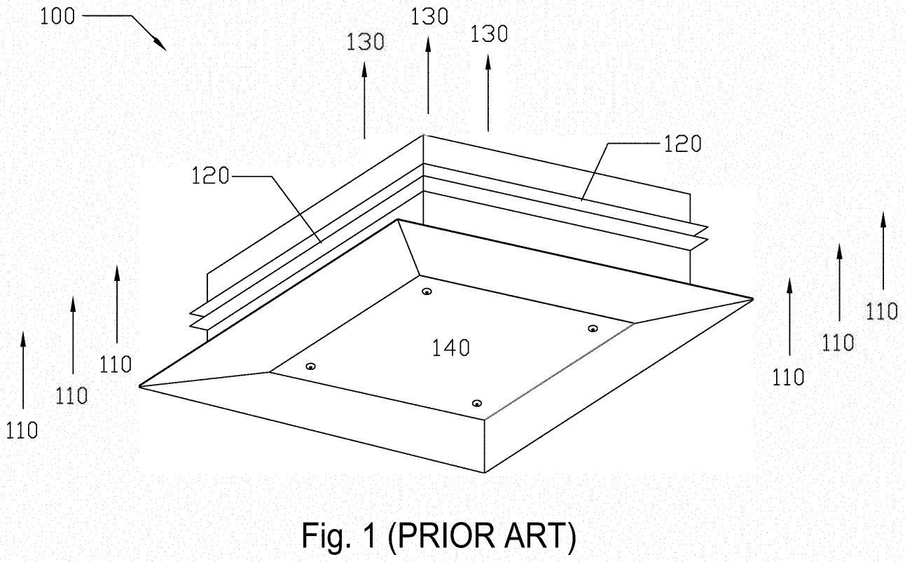

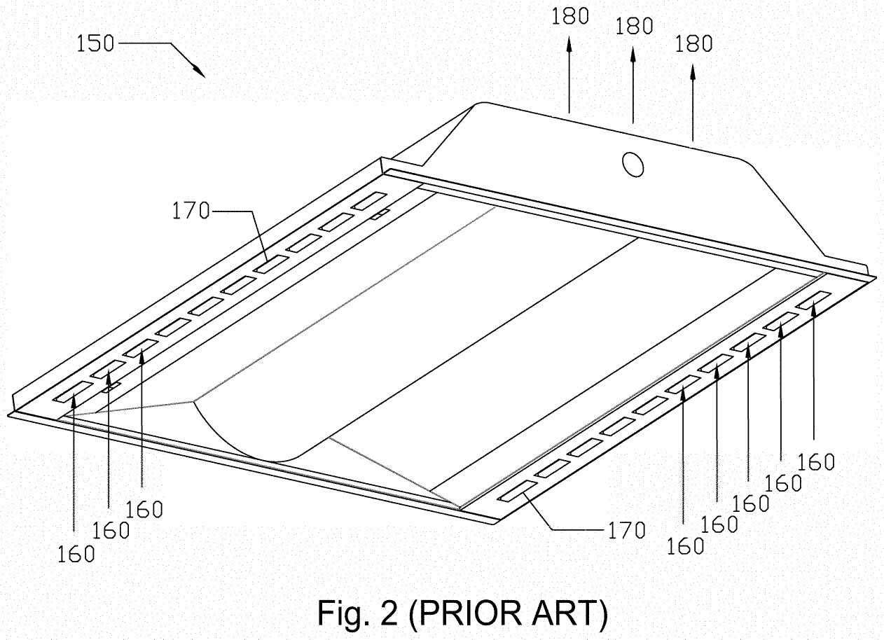

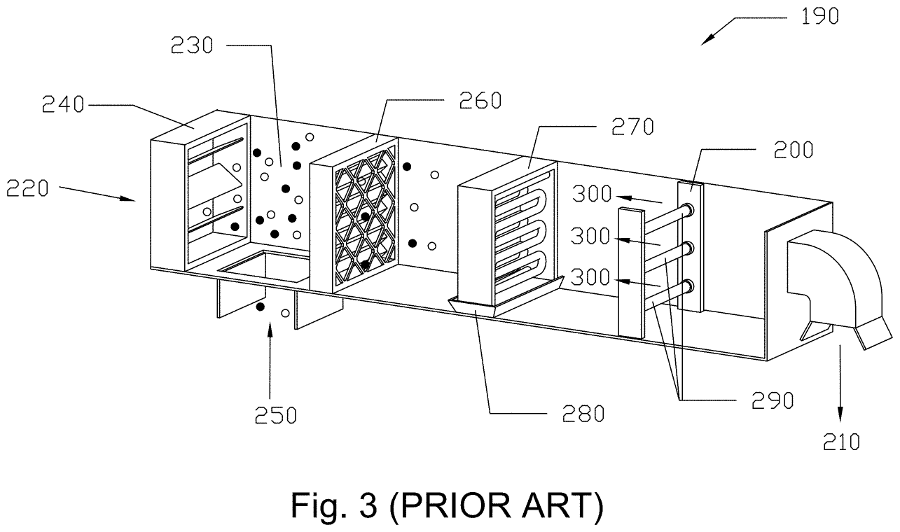

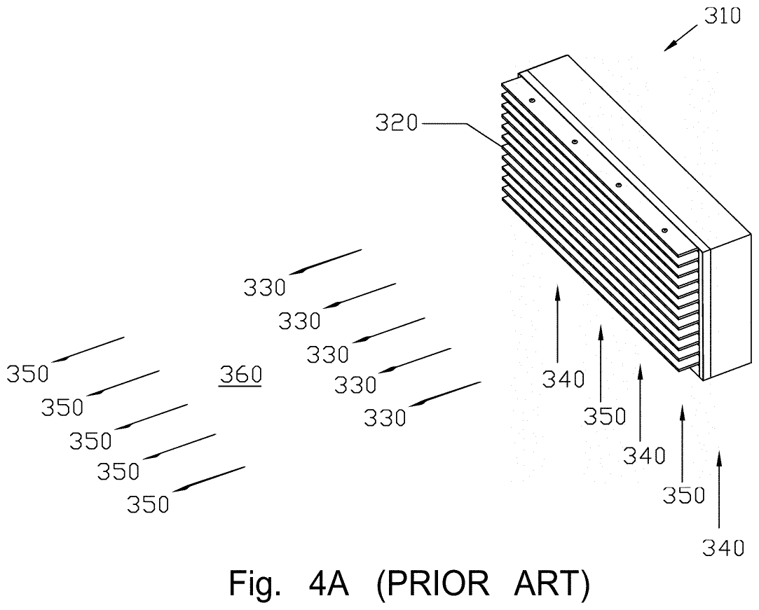

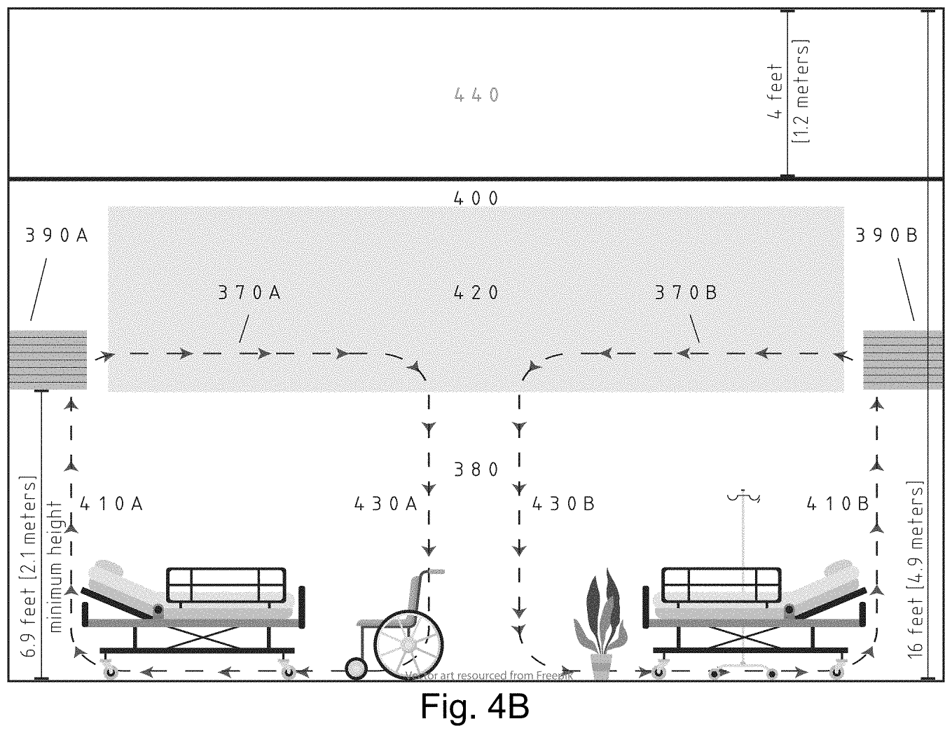

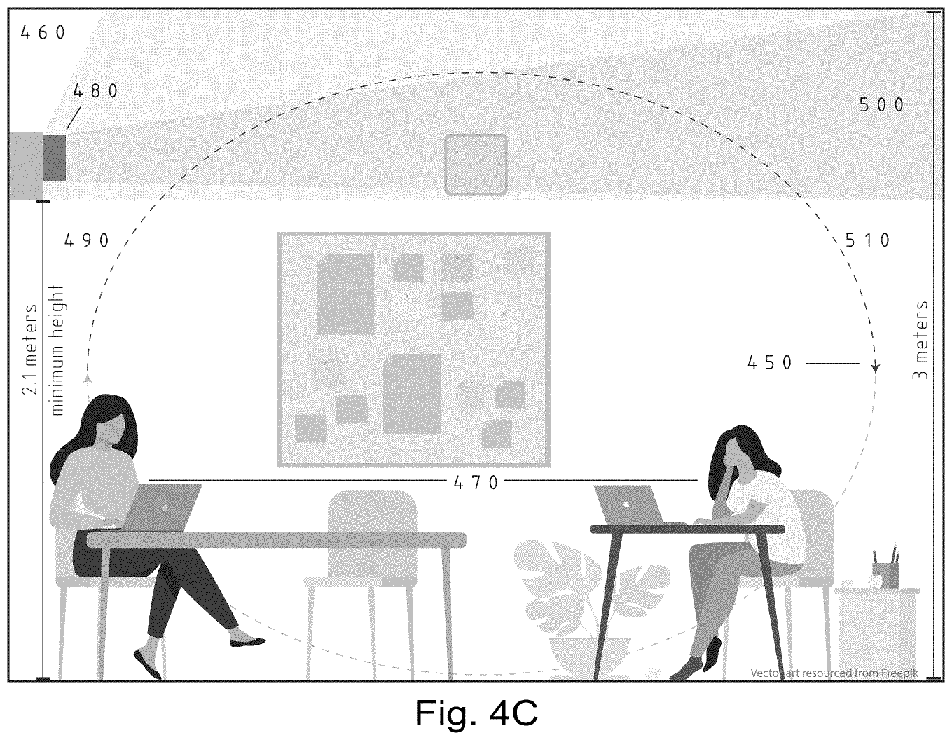

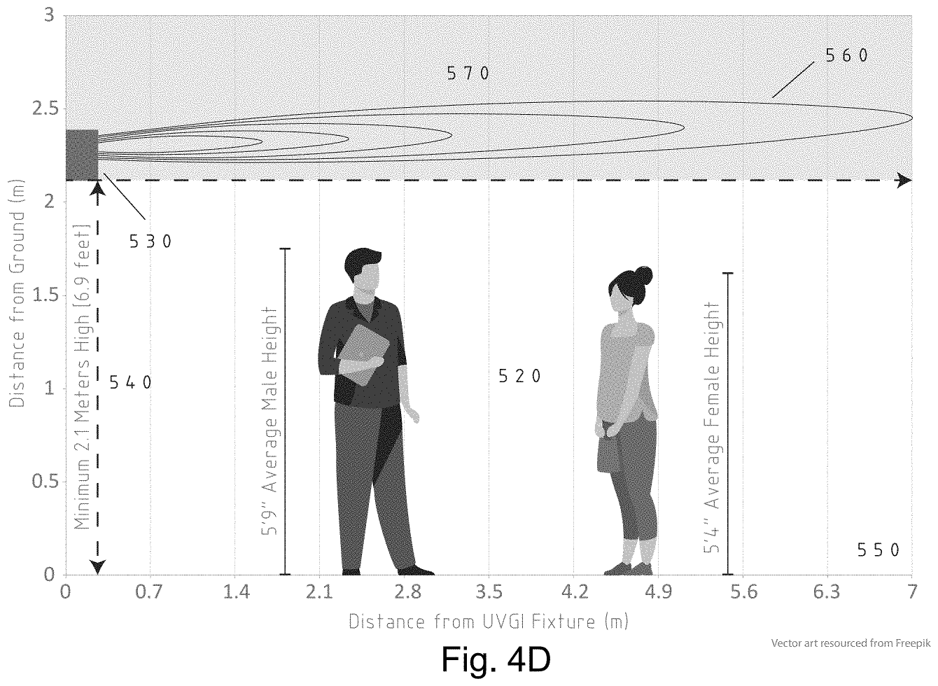

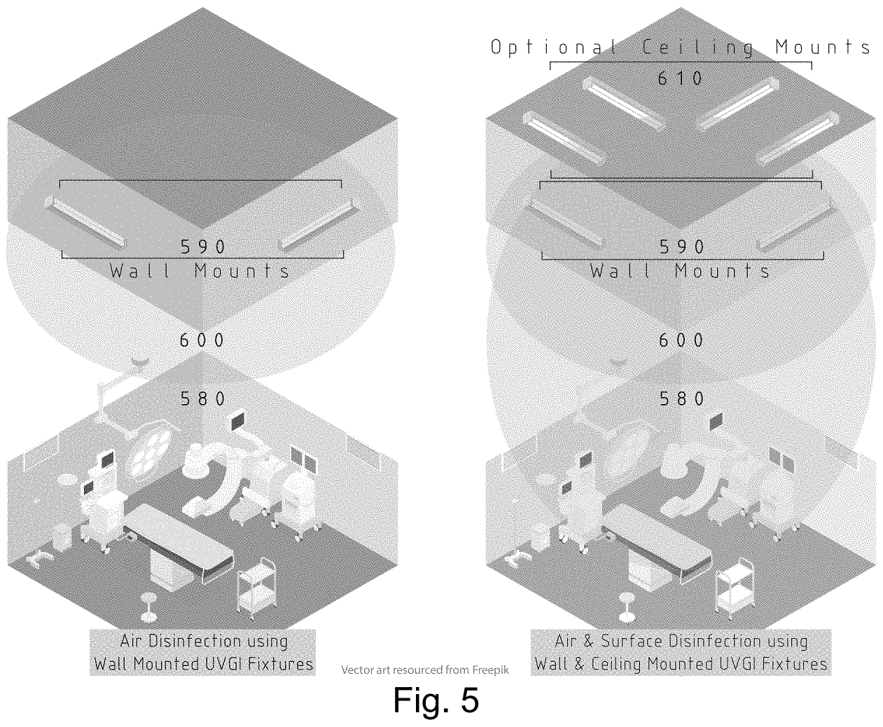

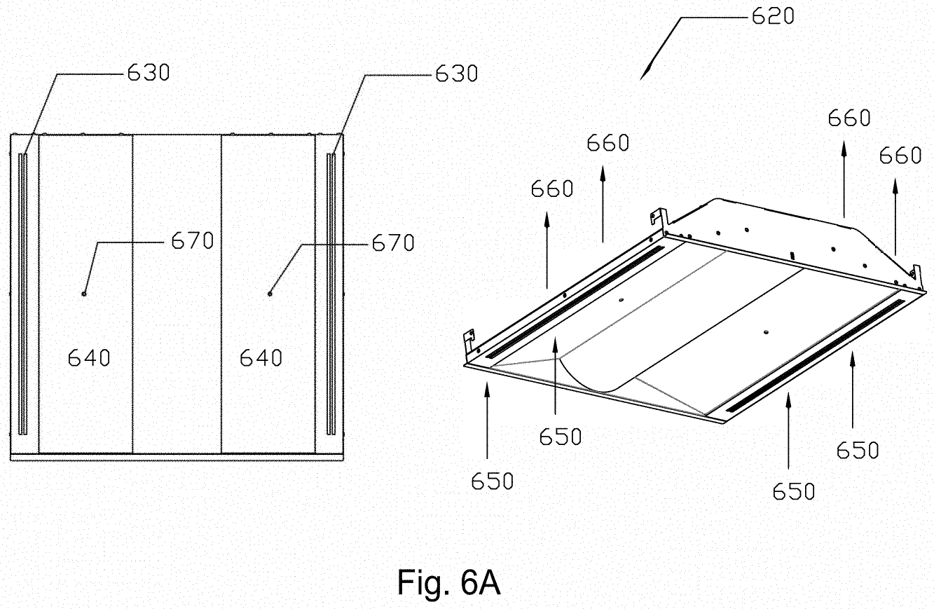

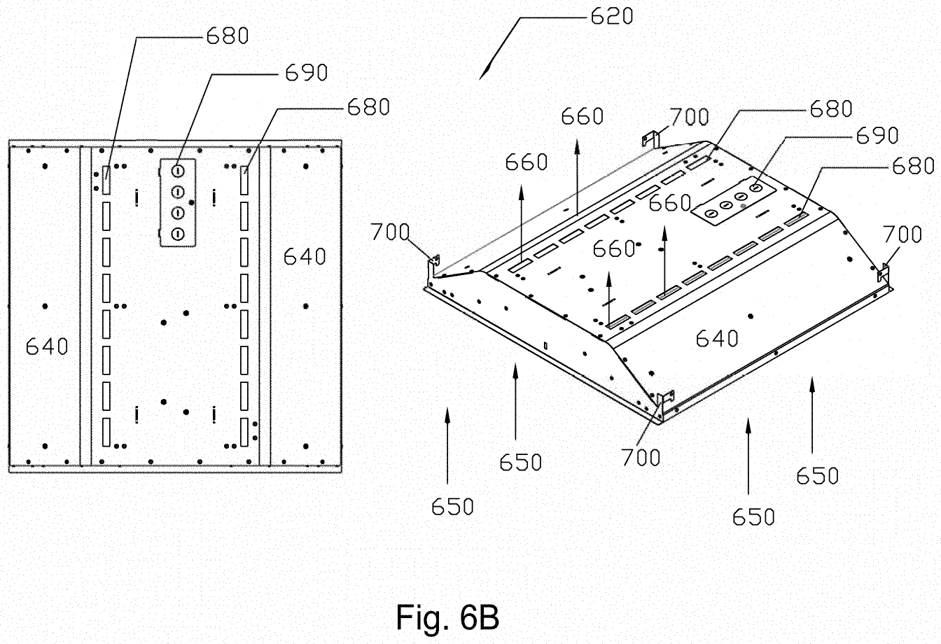

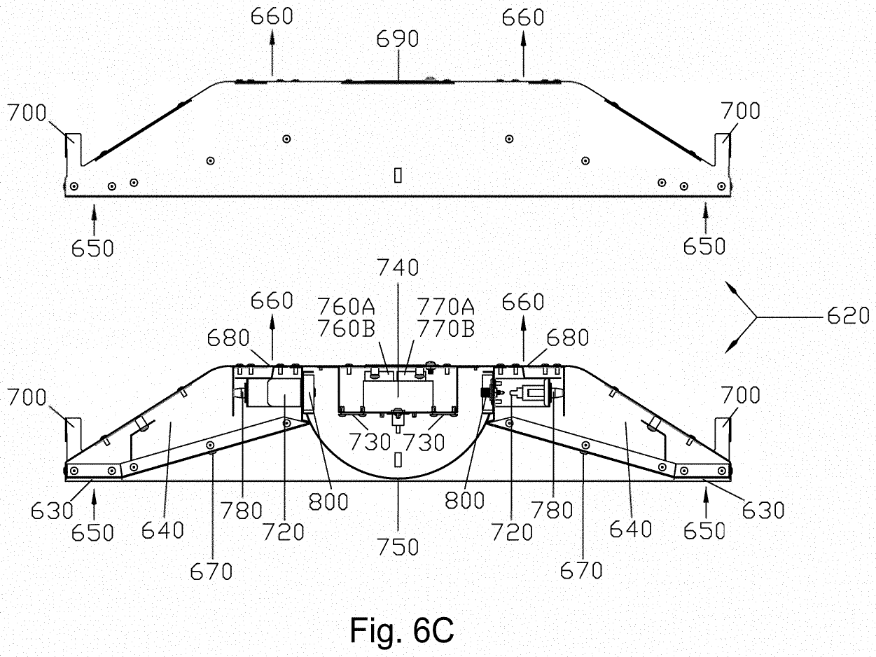

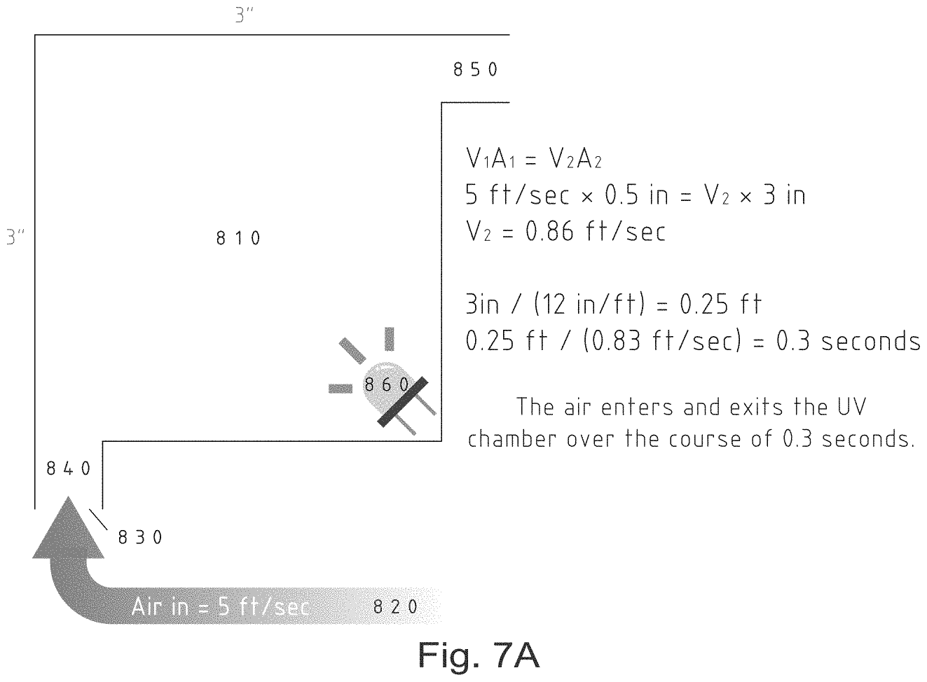

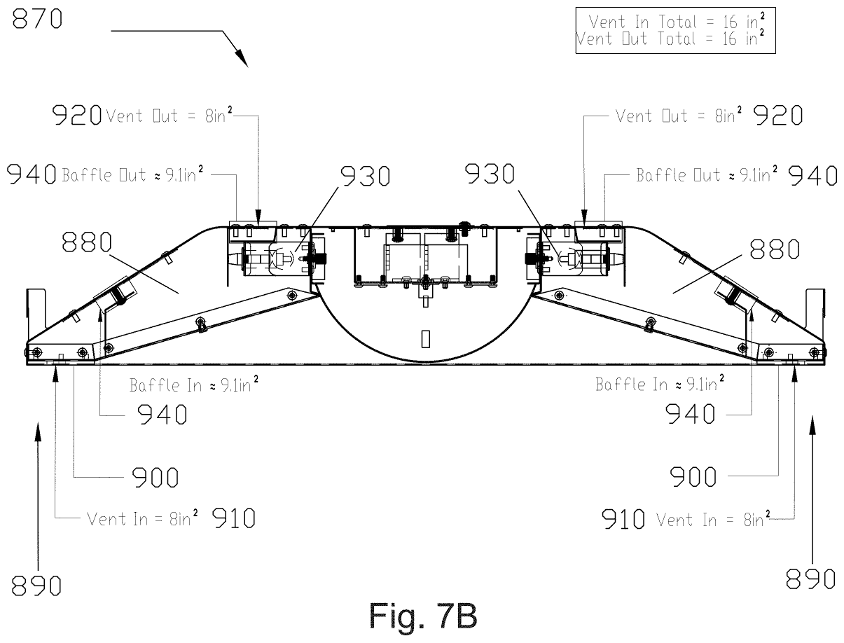

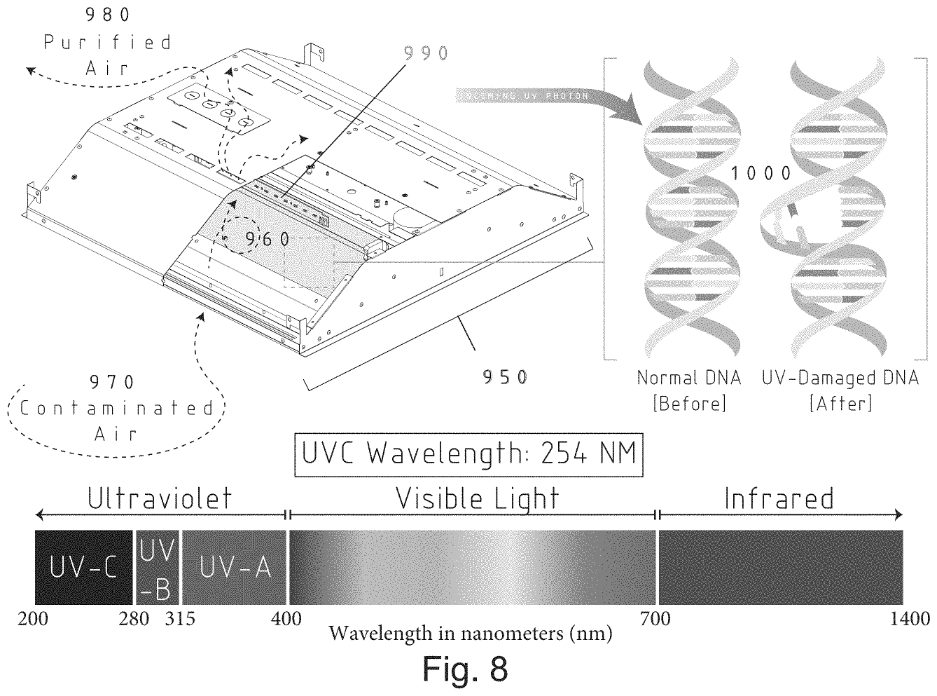

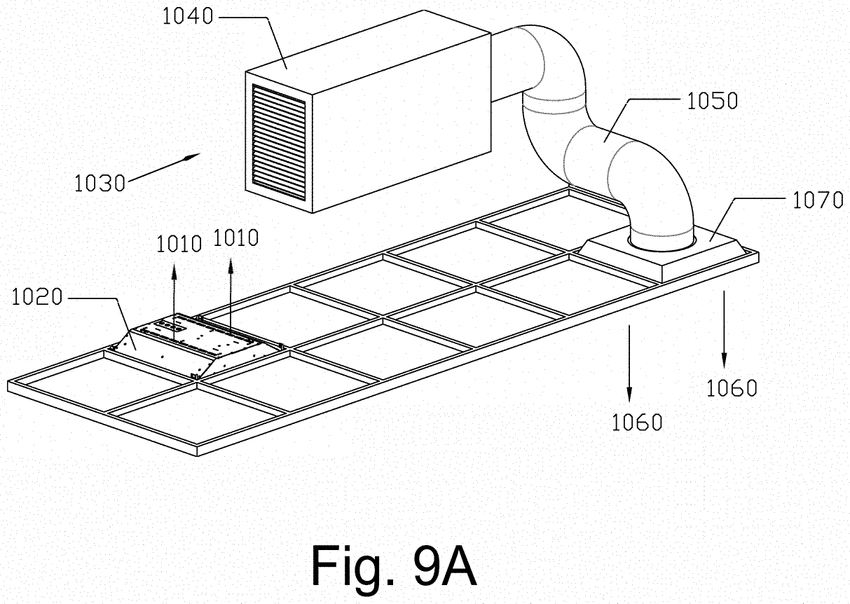

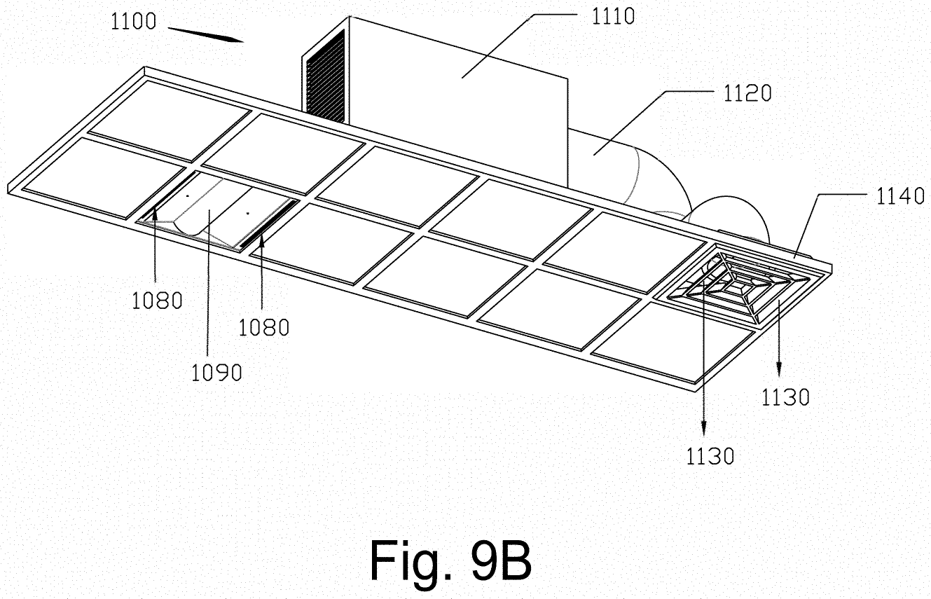

FIG. 1
100 air vent grill fixture
110 used air
120 air vent grills
130 return air
140 UVGI integrated cleaner
FIG. 2
150 air-handling troffer
160 used air
170 air vents
180 return air
FIG. 3
190 HVAC air duct
200 UVGI system
210 cool air
220 warm outside air
230 contaminants
240 mixing damper
250 return air
260 air filter
270 cooling coils
280 drain pan
290 UVC light sources
300 UVC light
FIG. 4A
310 UVGI fixture
320 light baffles
330 UVC light
340 used contaminated air
350 clean sanitized air
360 UVC disinfection zone
FIG. 4B
370A air flow 1
370B air flow 2
380 hospital room
390A UVGI fixture 1
390B UVGI fixture 2
400 ceiling
410A used contaminated air 1
410B used contaminated air 2
420 UVC disinfection zone
430A clean treated air 1
430B clean treated air 2
440 plenum space
FIG. 4C
450 air flow
460 meeting room
470 occupants
480 UVGI fixture
490 used contaminated air
500 UVC disinfection zone
510 clean treated air
FIG. 4D
520 meeting space
530 UVGI fixture
540 height
550 distance
560 UVC light
570 UVC disinfection zone
FIG. 5
580 operating room
590 wall mount UVGI fixtures
600 UVC disinfection zone
610 ceiling mount UVGI fixtures
FIG. 6A
620 LED light troffer and air-sanitizing fixture
630 air intake slot vents
640 internal UVGI air chamber(s)
650 used contaminated air
660 clean treated air
670 clear lens UVC light inspection window
FIG. 6B
680 air exhaust slot vents
690 power connection plate
700 metal clips
FIG. 6C
720 UVC light source
730 LED troffer light source
740 center basket
750 white color LED circuit boards
760A LED driver 1
760B LED driver 2
770A UVC light source power supply 1
770B UVC light source power supply 2
780 power disconnect switch
790 LED troffer light lens cover
800 lamp change panel
FIG. 7A
810 UVGI air chamber
820 contaminated air
830 air intake slot vents
840 air inlet area
850 air outlet area
860 UV light source
FIG. 7B
870 LED troffer light and air-sanitizing fixture
880 UVGI air chamber
890 contaminated air
900 air intake slot vents
910 air inlet area
920 air outlet area
930 UV light source
940 walls, baffles, or louvers
FIG. 8
950 disinfection and purification process
960 UVGI air chamber
970 used contaminated air
980 clean purified air
990 UVC germicidal light source
1000 bacteria, viruses, RNA and DNA
FIG. 9A
1010 clean treated return air
1020 LED light troffer and air-sanitizing fixture
1030 plenum space
1040 HVAC system
1050 ductwork
1060 room conditioned air
1070 vent grill
FIG. 9B
1080 used contaminated air
1090 LED light troffer and air-sanitizing fixture
1100 plenum space
1110 HVAC system
1120 ductwork
1130 room conditioned air
1140 vent grill

### DETAILED DESCRIPTION

FIG. 1 shows a drawing of an existing UVGI air vent grill fixture 100 that measures approximately 12″×12″. Used air 110 is sucked up into the side air vent grills 120 on all sides, cleaned, and then expelled into the plenum (not shown) as return air 130. This standalone device UVGI air vent grill fixture 100 simply replaces a regular passive air return vent with the addition of an internal UVGI integrated cleaner 140 inside UVGI air vent grill fixture 100.

FIG. 2 shows a drawing of an existing air-handling troffer 150. Such a fixture is available from ILP Inc. under their Airvola Troffer brand models. The Airvola recessed air-handling troffers are available in 2′×2′ and 2′×4′ sizes. Used air 160 is sucked up into the bottom air vents 170 located on either side of the air-handling troffer 150, and exits out on top as return air 180. There are shown ten air vent openings 170 on either side of the air-handling troffer 150 for a total of twenty air vent openings 170 in total for a 2′×2′ size troffer. This recessed air-handling troffer 150 device simply adds the feature of return air 180 back to the HVAC system (not shown), but does nothing to disinfect the used air 160 in a room as it has no UVGI or Ultraviolet Germicidal Irradiation means.

FIG. 3 shows a drawing of a typical exploded view of the inside portion of a HVAC air-duct 190 with a UVGI system 200 used to clean the air to a building. In particular, the drawing shows how cool air 210 is conditioned and treated before it is supplied to a building. Warm outside air 220 contains mold spores, bacteria, viruses, and VOC's contaminants 230. A mixing damper 240 combines the warm outside air 220 with the return air 250 from the building that may also contain some mold spores, bacteria, viruses, and VOC's contaminants 230. The mixed warm outside air 220 and return air 250 is sent through an air filter 260 to remove some of the contaminants 230 onto cooling coils 270. Mold spore contaminants 230 may reproduce in the air filter 260, cooling coils 270, and drain pan 280. An array of UVC light sources 290 is part of the UVGI system 200 producing UVC light 300 to destroy the mold spores, bacteria, viruses, and VOC's contaminants 230 preventing them from entering the cool air 210. Lastly, the clean and treated cool air 210 supply is sent to the building ventilation system (not shown).

FIG. 4A shows a drawing of a high wall mounted upper air UVGI fixture 310. The upper air UVGI fixture 310 can be portable or permanently installed on a wall (not shown) high up close to the ceiling. There are twelve light baffles 320 positioned in front of the upper air UVGI fixture 310 to prevent the UVC light 330 to be visible or shine downwards below upper air UVGI fixture 310 for safety. There are intake openings (not shown) located on the bottom of the upper air UVGI fixture 310 that suck up mostly used contaminated air 340 from a room using internal fans (not shown). The mostly used contaminated air 340 is cleaned and sanitized in the upper area of the room called the UVC disinfection zone 360 where the UVC light 330 is most concentrated. Forced air flow out from the front facing exhaust openings (not shown) will circulate the air straight out as clean sanitized air 350, and then downwards where natural convection will send the clean sanitized air 350 to again mix with the mostly used contaminated air 340 both back up into air intake openings (not shown) located on the bottom of the upper air UVGI fixture 310 for continuous cleaning and sanitizing in the UVC disinfection zone 360 when the upper air high wall mounted UVGI fixture 310 is energized.

FIG. 4B shows a picture of the air flow 370A, 370B from a typical high wall mounted upper air UVGI fixture 310 shown in FIG. 4A used now in a typical hospital room 380. Double wall mounted upper air UVGI fixtures 390A, 390B are installed on opposite ends of the hospital room 380 each showing how the continuous air flow 370A, 370B both push the contaminated used air 410A, 410B up to in the UVC disinfection zone 420, and the clean treated air 430A, 430B is recycled back into each wall mounted upper air UVGI fixture 390A, 390B respectively located on either side of a typical hospital room 380. Note the optimum location of each wall mounted upper air UVGI fixture 390A, 390B are installed at a minimum height of 6.9 feet or 2.1 meters with a ceiling 400 plenum space 440 shown here with a relative height of 4.0 feet or 1.2 meters, and the overall height of the typical hospital room 380 at 16.0 feet or 4.9 meters high.

FIG. 4C is an alternate picture showing the air flow 450 from the high wall mounted upper air UVGI fixture 310 described in FIG. 4A, but now in a meeting room 460 filled with occupants 470. A single wall mounted upper air UVGI fixture 480 is installed showing how the continuous air flow 450 pushes the used contaminated air 490 up into the UVC disinfection zone 500, and the clean treated air 510 is recycled back into the wall mounted upper air UVGI fixture 480 located on one side of meeting room 460.

FIG. 4D shows a picture of a meeting space 520 with one high wall mounted upper air UVGI fixture 530 installed showing the minimum safe installation height 540 of the upper air UVGI fixture 530, and the maximum effective sterilization distance 550 from the upper air UVGI fixture 530 germicidal UVC light source 560. The upper air UVGI unit 530 is typically installed on a wall at a minimum height 540 of 2.1-meters or about 7.0-feet off the floor with an effective maximum distance 550 range of up to 7.0-meters or about 23.0-feet from the upper air UVGI fixture 530 with sanitizing UVC light 560 defining UVC disinfection zone 570.

FIG. 5 shows how a room can be cleaned using only wall mounted upper air UVGI fixtures for air disinfection, and also shows how the same room can be cleaned using a combination of wall mounted upper air UVGI fixtures and ceiling mounted UVGI fixtures for both air and surface disinfection. In particular FIG. 5 shows a typical operating room 580 can be cleaned using only wall mounted upper air UVGI fixtures 590 for air disinfection from the picture on the left. This operating room 580 can be occupied with only wall mounted upper air UVGI fixtures 590 installed as long as the occupants (not shown) are not anywhere near the upper UVC disinfection zone 600. The picture on the right shows the same operating room 580 can be cleaned using a combination of wall mounted upper air UVGI fixtures 590 and ceiling mounted UVGI fixtures 610 for both air and surface disinfection. With the addition of ceiling mounted UVGI fixtures 610, occupants (not shown) will not be permitted to occupy the operating room 580 when the ceiling mounted UVGI fixtures 610 are energized for their personal safety. They must be turned-off manually with a wall switch (not shown) or automatically using occupancy sensors (not shown).

FIG. 6A shows both a front and isometric view of the present invention of an improved LED light troffer combined with a UVGI air-sanitizing from the ground showing typical air intake slots or vents, etc. In particular, FIG. 6A shows both a front and isometric view of the present invention of an improved 2′×2′ LED light troffer combined with a UV air-sanitizing fixture 620 as seen from the ground showing typical air intake slot vents 630. The air intake slot vents 630 can be located on one or both sides of the troffer light 620 and can be one or more openings. The air intake slot vent 630 openings lead into an internal UVGI air chamber 640 (not shown) for the safe disinfection of the used contaminated air 650, which is sanitized by UVC light (not shown) within the internal UVGI air chamber 640 (not shown). There are two individual and separate internal UVGI air chambers 640 (not shown) located on either side of the LED light troffer and air-sanitizing fixture 620. The now clean treated air 660 is expelled out of the internal UVGI air chambers 640 (not shown) of LED troffer light and air-sanitizing fixture 620 and fed into the air return of the HVAC system (not shown). The total air return space is estimated at 12-½″ square for a 2′×2′ combined LED light troffer and UVGI air-sanitizing fixture 620, and 25-½″ square for a 1′×4′ and 2′×4′ combined LED light troffer and UVGI air-sanitizing fixture (not shown). A clear lens UVC light inspection window 670 is located on both sides of the LED light troffer and air-sanitizing fixture 620 to show the presence of UVC light (not shown) in each internal UVGI air chamber 640 (not shown) for proper air-sanitizing activity. Clear lens UVC light inspection window 670 can be seen from below the ceiling at a safe distance away from the UVC light (not shown) that is contained within each internal UVGI air chamber 640 (not shown) of LED light troffer and air-sanitizing fixture 620.

FIG. 6B shows both a rear and isometric view of the present invention of an improved LED light troffer combined with a UVGI air-sanitizing from the ground showing typical air intake slots or vents, etc. In particular, FIG. 6B shows both a rear and isometric view of the present invention of an improved 2′×2′ LED light troffer combined with a UV air-sanitizing fixture 620 as seen from above the ceiling of the same LED light troffer and UV air-sanitizing fixture 620 of FIG. 6A now showing typical air exhaust slot vents 680. The air exhaust slot vents 680 can be located on one or both sides of the troffer light 620 and can be one or more openings. The air exhaust slot vents 680 openings lead out from an internal UVGI air chamber 640 (not shown) for the expulsion of clean treated air 660, which is sanitized by UVC light (not shown) within the internal UVGI air chamber 640 (not shown). There are two individual and separate internal UVGI air chambers 640 (not shown) located on either side of the troffer and air-sanitizing fixture 620. The clean treated air 660 exits out of the internal UVGI air chambers 640 (not shown) of LED troffer light and air-sanitizing fixture 620 and is fed into the air return of the HVAC system (not shown). Main power connection to the LED light troffer and air-sanitizing fixture 620 is accomplished by utilizing power connection plate 690 and performed by a qualified electrician. Two individual and separate power circuits will be connected to the LED light troffer and air-sanitizing fixture 620. One to power the LED troffer general illumination lights and another to power the UVC light sources in the internal UVGI air chambers 640 (not shown). In addition, four metal clips 700 are provided for mounting and installation of the LED light troffer and air-sanitizing fixture 620 to a lighting T-grid or drop ceilings (not shown).

FIG. 6C show side views of the present invention of an improved LED light troffer combined with a UVGI air-sanitizer with the end plate present and also removed showing typical air intake slots or vents and internal parts. In particular, FIG. 6C shows side views of the present invention of an improved 2′×2′ LED light troffer combined with a UV air-sanitizing fixture 620 as seen from the sides when installed above the ceiling of the same LED light troffer and UV air-sanitizing fixture 620 of FIG. 6A. Air intake slot vents 630 and air exhaust slot vents 680 are be located on one or both sides of the troffer light 620 and can be one or more openings. The air intake slot vent 630 openings lead into the internal UVGI air chamber 640 for the safe disinfection of the used contaminated air 650, which is sanitized by UVC light source 720 within the internal UVGI air chamber 640. There are two individual and separate internal UVGI air chambers 640 located on either side of the LED light troffer and air-sanitizing fixture 620. The clean treated air 660 is expelled out of the internal UVGI air chambers 640 through the air exhaust slot vents 680 of LED troffer light and air-sanitizing fixture 620 and is fed into the air return of the HVAC system (not shown). Main power connection to the LED light troffer and air-sanitizing fixture 620 is accomplished by utilizing power connection plate 690 and performed by a qualified electrician. Two individual and separate power circuits will be connected to the LED light troffer and air-sanitizing fixture 620. One to power the LED troffer general illumination lights and another to power the UVC light sources in the internal UVGI air chambers 640. In addition, four metal clips 700 are provided for mounting and installation of the LED light troffer and air-sanitizing fixture 620 to a lighting T-grid or drop ceilings (not shown). LED troffer light source 730 is located in the center basket 740 of the LED light troffer and UV air-sanitizing fixture 620, consisting mainly of white color LED circuit boards 750 for general illumination that are powered by LED drivers 760A, 760B located under the center basket 740. UVC light source 720 located in each internal UVGI air chamber 640 can be any source of UVC light including UVC germicidal fluorescent tube lamps, or cold cathode UVC tubes, UVC excimer light sources in addition to UVC LEDs (not shown). Each UVC light source 720 is powered by a UVC light source power supply 770, which may also reside in the center basket 740. In the event when either clear lens UVC light inspection window 670 shows no light from UVC light source 720 is present, the UVC light source 720 will need to be replaced. For electrical safety during routine maintenance and UVC light source 720 replacement, power disconnect switch 780 is used in each internal UVGI air chamber 640 to turn off power to each UVC light source power supply 770A, 770B to de-energize each UVC light source 720 when either lamp change panel 800 is opened to access the internal UVGI air chamber 640.

FIG. 7A shows an inside view of an exemplary embodiment of a UVGI air chamber of the present invention showing air entering from the intake slots or vents from FIG. 6A. In particular, FIG. 7A shows a top view of one exemplary embodiment of a UVGI air chamber 810 of the present invention showing contaminated air 820 entering from the air intake slot vents 830. The UVGI air chamber 810 shown for sanitizing is 3″×3″ with a ½ in² air inlet area 840 and a ½ in² air outlet area 850. For minimal air flow noise, the air velocity coming into the UVGI air chamber 810 should be about 5 feet per second. For the Bernoulli Equation of Continuity V₁A₁=V₂A₂: V₁=Air Velocity in; V₂=Air Velocity out; A₁=Area of Opening in; and A₂=Area of Opening out. Now solving for Air Velocity out V₂, we then have 5*0.5=V₂*3=0.83 feet per second. Therefore, the air flow will slow down while in the UVGI air chamber 810 for [(3″/12″ feet)=0.25 feet/0.83 feet per second]=0.3 seconds, and the contaminated air 820 has to be treated and cleaned during this brief amount of time. Other methods of slowing down the air flow inside the UVGI air chamber 810 would be to use a maze of obstruction walls, baffles, or louvers (not shown) at the output of the UVGI air chamber 810 that would give the UV light source 860 more time to sanitize the contaminated air 820.

FIG. 7B shows a side view of a typical embodiment of a UVGI air chamber of the present invention showing air passing through the slots or vents from FIGS. 6A-6C. In particular, FIG. 7B is a cross-sectional transparent end view of a LED light troffer and air-sanitizing fixture 870 depicting a UVGI air chamber 880 located to the left and right sides of the present invention showing contaminated air 890 entering from the air intake slot vents 900. Each UVGI air chamber 880 shown for sanitizing has an 8 in² air inlet area 910 and an 8 in² air outlet area 920. By creating an expanded 24″ long UVGI air chamber 880 inside both sides of the LED troffer light and air-sanitizing fixture 870 for UV disinfection, the velocity of the air is significantly reduced increasing dwell time in each UVGI air chamber 880. This significantly reduces the UV power required to achieve the proper UV dose needed for disinfection. For minimal air flow noise, the air velocity coming into the UVGI air chamber 880 should be about 5 feet per second. For the Bernoulli Equation of Continuity V₁A₁=V₂A₂: V₁=Air Velocity in; V₂=Air Velocity out; A₁=Area of Opening in; and A₂=Area of Opening out. Now solving for Air Velocity out V₂, we then have 5*8=V₂*24=0.6 feet per second. Therefore, the air flow will slow down while in the UVGI air chamber 880 for [(24″/12″ feet)=2.0 feet/0.6 feet per second]=3.33 seconds, and the contaminated air 890 has to be treated and cleaned during this brief amount of time. Other methods of slowing down the air flow inside the UVGI air chamber 880 would be to use a maze of obstruction walls, baffles, or louvers 940 at the output of the UVGI air chamber 880 that would give the UV light source 930 more time to sanitize the contaminated air 890.

FIG. 8 shows a picture of the disinfection and purification process taking place within the UVGI air chamber of FIG. 7A where the used contaminated air enters and the clean treated air exits the LED light troffer and UVGI air-sanitizing fixture. In particular, FIG. 8 shows a picture of the disinfection and purification process 950 taking place within the UVGI air chamber 960 when the used contaminated air 970 enters and the clean purified air 980 exits. Used contaminated air 970 enters the UVGI air chamber 960 from the bottom vents (not shown), is clean and treated by the UVC germicidal light source 990 to deactivate or kill bacteria, viruses, RNA, and DNA 1000, and clean purified air 980 is then released out of the UVGI air chamber 960. Ultraviolet light has wavelengths from 200-400 nm with the most effective UVC wavelength at 254 nm. Visible light is from 400-700 nm, and infrared light ranges from 700-1400 nm.

FIG. 9A shows a top view above the ceiling of the now clean and treated return air exiting the UVGI air chamber as described in FIG. 8 of the present invention next entering the return air circulation and plenum space, and then back to the main HVAC system for recycling using a standard ductwork and air grill. In particular, FIG. 9A shows the now pure clean treated return air 1010 exiting the UVGI air chamber (not shown) as described in FIG. 8 of the present invention of an LED light troffer and air-sanitizing fixture 1020 next entering the return air circulation and plenum space 1030, and then back to the main HVAC system 1040 for recycling using standard ductwork 1050 providing room conditioned air 1060 entering back into a room (not shown) through vent grill 1070.

FIG. 9B shows a bottom view below the ceiling of the now clean and treated return air exiting the UVGI air chamber as described in FIG. 8 of the present invention next entering the return air circulation and plenum space, and then back to the main HVAC system for recycling using a standard ductwork and air grill. In particular, FIG. 9B shows the used contaminated air 1080 entering the UVGI air chamber (not shown) as described in FIG. 8 of the present invention of an LED light troffer and air-sanitizing fixture 1090 next entering the return air circulation and plenum space 1100, and then back to the main HVAC system 1110 for recycling using standard ductwork 1120 providing room conditioned air 1130 entering back into a room (not shown) through vent grill 1140.

It will be understood that various changes in the details, materials, types, values, and arrangements of the components that have been described and illustrated in order to explain the nature of this invention may be made by those skilled in the art without departing from the principle and scope of the invention as expressed in the following claims.

What is claimed is:

1. An improved lighting troffer for installation in a ceiling in a building with an HVAC system comprising:
a visible light portion,
an ultraviolet light portion embedded within said lighting troffer,
wherein said ultraviolet light portion includes air input openings to take in used air from the room space below said ceiling,
wherein said ultraviolet light portion irradiates and sanitizes said used air,
wherein said ultraviolet light portion further includes air output openings to expel cleaned air out into the plenum space above said ceiling, and
the treated air is returned back to the said HVAC system and recycled.

2. The improved lighting troffer according to claim 1, wherein said visible light portion is generated by at least one linear white color LED circuit board.

3. The improved light troffer according to claim 2, wherein said at least one linear white color LED circuit board is mounted to a center basket frame.

4. The improved lighting troffer according to claim 3, further including:
at least one LED driver mounted in juxtaposition to and providing power to at least one linear white color LED circuit board mounted to said center basket frame.

5. The improved light troffer according to claim 1, wherein said ultraviolet light portion includes:
at least one UVC air chamber containing a UVC light source.

6. The improved light troffer according to claim 5, wherein UVC light source is a UVC germicidal fluorescent lamp.

7. The improved light troffer according to claim 5, wherein UVC light source is a UVC cold cathode lamp.

8. The improved light troffer according to claim 5, wherein UVC light source is a UVC excimer type lamp.

9. The improved light troffer according to claim 5, wherein UVC light source is at least one UVC type LED emitter.

10. The improved light troffer according to claim 5, further including:
a separate power source to provide power to said UVC light source in said at least one UVC air chamber.

11. The improved light troffer according to claim 5, further including:
at least one inspection window for the presence of UVC light from said UVC light source that can be easily seen from below the ceiling.

12. A lighting fixture comprising:
a plurality of general illumination light sources for providing visible light to an occupied space,
at least one ultraviolet germicidal irradiation air chamber juxtaposed to said plurality of general illumination light sources with air intake and air exhaust vent openings,
wherein the used air from said occupied space enters said at least one ultraviolet germicidal irradiation air chamber by way of said air intake and is sanitized by an ultraviolet light source,
wherein the cleaned air exits said at least one ultraviolet germicidal irradiation air chamber by way of said air exhaust, and
the treated air is returned back to the main heating ventilation air conditioning system to be recycled.

13. A device for use in a lighting fixture installed in a building with an HVAC system comprising:
an ultraviolet light air chamber with input and output openings,
wherein said ultraviolet light air chamber allows used air to pass thru said input opening without impediment of the at least 500 fpm moving airflow,
wherein said ultraviolet light air chamber further irradiates said used air with a target dose of ultraviolet light of at least 1,500 uW/cm² at an exposure time of at least 0.25 seconds, and at an irradiance zone of at least twenty-four inches, and
wherein said ultraviolet light air chamber allows cleaned air to pass thru said output opening into the plenum space and back into the HVAC system to be recycled.
